(19) United States
(12) Patent Application Publication
(10) Pub. No.: US 2026/0215964 A1
(43) Pub. Date: Jul. 30, 2026

(54) DOCKING STATION FOR USE IN OPHTHALMIC PROCEDURES

(71) Applicants: Horizon Surgical Systems, Inc., Malibu, CA (US); The Regents of the University of California, Oakland, CA (US)

(72) Inventors: Matthew GERBER, Tokyo (JP); Peter FERGUSON, Los Angeles, CA (US); Tyler KRAUSS, Los Angeles, CA (US); Jean-Pierre HUBSCHMAN, Malibu, CA (US); Jacob ROSEN, Los Angeles, CA (US)

(21) Appl. No.: 19/146,201

(22) PCT Filed: Jan. 5, 2024
(86) PCT No.: PCT/US2024/010589

§ 371 Date: Jul. 7, 2025

Related U.S. Application Data
(60) Provisional application No. 63/478,851, filed on Jan. 6, 2023.

(51) Int. Cl. A61F 9/007; A61B 17/00; A61B 90/30; A61B 90/50; A61F 9/00

(52) U.S. Cl. CPC A61F 9/007; A61B 17/00; A61B 90/30; A61B 90/50; A61F 9/0026; A61B 2017/00477; A61F 2009/0052

(57) ABSTRACT

There is a docking device for intraocular surgery having an upper rim having a second diameter, a lower rim having a first diameter and a bottom surface with at least one aperture, and a sidewall extending between the upper rim and the lower rim. There is also an arm having a first portion coupled to a central portion of the upper rim and the lower rim and a second portion configured for connection to a positioning arm. The lower rim also has a lower surface having an adapted contour configured to be secured to the sclera and/or conjunctiva of a procedure eye. Optionally, the lower surface of the upper rim is adapted and configured for releasable engagement with an upper surface of the lower rim. Other variations include a breakaway coupling allowing the suction surface to remain coupled to the procedure eye with other portions of the docking device removed.

### CLAIM OF PRIORITY

This application claims priority to U.S. Provisional Application No. 63/478,851, titled “DOCKING STATION FOR USE IN OPHTHALMIC PROCEDURES,” filed Jan. 6, 2023, the contents of which is incorporated herein by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This application relates to systems and tools for use as an interface during ophthalmic procedures.

### BACKGROUND

Intraocular medical conditions are treated with delicate microsurgical procedures that rely on visualization of delicate tissues within a small and constrained space of an eye. Optical coherence tomography (OCT) can be used to improve intraocular visualization by imaging small membranes and anatomical features to reveal features that would otherwise remain invisible or difficult to perceive by a surgeon, thereby improving surgical outcomes. In addition, OCT is capable of providing fast, small-scale, and accurate measurements which allow for the possibility of real-time guidance to the surgeon or to an autonomous robotic surgical system. In addition to OCT, a surgical microscope can be used to visualize features inside the eye. In addition to imaging devices, laser-based manipulation devices such as femtosecond laser systems can be used to cut, slice, or physically change the eye.

Despite the advantages of OCT systems, surgical microscopes, and laser-based surgical devices, the quality of both imaging and light-based manipulation systems can suffer from the nature of a surgical environment. First, the constant motion either from the patient movement or when surgical tools are manipulated inside the eyeball can result in high levels of noise, data distortion, and physical inaccuracy within the visualized area. Second, OCT scan quality in particular can be dependent on the presence of a fluid medium between an imaging probe and anatomy to be scanned; therefore, presence of a fluid medium can maintain eye hydration in addition to improving visualization quality. Further, the imaging system is located in a patient sterile field but cannot itself be adequately sterilized, thereby constraining its use and integration into other systems, such as robotic surgical systems. In addition, while docking systems may exist, none are capable of simultaneously allowing for a surgical instrument to manipulate the eye when the docking is engaged. Allowing simultaneous docking and instrument manipulation would provide some or all of the abovementioned benefits without comprising the need to perform surgical manipulation.

Additional challenges are presented with regard to stabilization of the eye generally. First, there is largely unconstrained patient eye motion during an intraocular procedure. Second, the eye is subject to movement and displacement due to surgical tool forces and torques during a surgical procedure. Third, unconstrained motion is an impediment to successful implementation of robotically assisted or fully robotic applications and can decrease the quality of visualization of the intraocular workspace. Eye motion while surgical instruments are inside the eye can lead to severe and/or irreversible damage to the eye anatomy. These factors highlight an additional unmet requirement for eye stability in a docking system.

It is against this background that the need for continued improvement in the field of eye stabilization on systems suited to advanced and emerging intraocular surgical systems and techniques.

### SUMMARY OF THE DISCLOSURE

In various embodiments, there is described a docking device for intraocular surgery having an upper rim having a second diameter; a lower rim having a first diameter and a bottom surface with at least one aperture; an arm having a first portion coupled to a central portion of the upper rim and the lower rim and a second portion configured for connection to a positioning arm; and a lumen within or along the arm in communication with the at least one aperture. In additional aspects, a first end of the upper rim and a second end of the upper rim define an opening from 30 degrees to 180 degrees. In additional embodiments, the first diameter is the same as the second diameter. Additionally or optionally, the first diameter is greater than the diameter of the corneal limbus of a procedure eye. In other embodiments, the first diameter is less than the second diameter. In one aspect, the lower rim further comprising a lower surface having a curvature, a contour or a durometer adapted and configured to be secured to the sclera and/or conjunctiva of a procedure eye. In another embodiment, a lower surface of the upper rim is adapted and configured for releasable engagement with an upper surface of the lower rim. In still additional aspects, there is an opening between the central portion and the first end of the upper rim or between the central portion and the second end of the upper rim.

The various alternative embodiments also provide for one or more methods of stabilizing an eye during an ophthalmic procedure. In one embodiment, there is a step of positioning a lower surface of a suction ring on the eye such that a central portion of the suction ring is at, on, or adjacent to a plica semilunaris and a lacrimal caruncle and a portion of an opening in the suction ring is opposite to the central portion, the opening ranging from 30 degrees to 180 degrees. Next, there is a step of applying vacuum to the suction ring to secure the suction ring lower surface to the surface of the sclera and/or conjunctiva, the suction ring at least partially encircling the cornea of the eye. There is also a step of performing the ophthalmic procedure on the eye using an instrument passed through the opening in the suction ring. In additional embodiments, there is also a step of operating a positioning arm of an ophthalmic imaging and illumination system prior to the positioning step and the applying the vacuum step so that the suction ring is in alignment for use with the ophthalmic imaging and illumination system and for providing access to the eye using the opening in the suction ring. In still other embodiments there is a step of prior to the operating a positioning arm step releasably coupling the suction ring to the positioning arm.

Additionally, the above methods may be modified wherein moving a patient head during the ophthalmic procedure will uncouple the suction ring from the positioning arm.

In still other alternative embodiments, there is provided a docking device for intraocular surgery having an upper rim having a second diameter, a lower rim having a first diameter and a bottom surface with at least one aperture, and a sidewall extending between the upper rim and the lower rim. There is also an arm having a first portion coupled to a central portion of the upper rim and the lower rim and a second portion configured for connection to a positioning arm. There is also a lumen within or along the arm in communication with the at least one aperture, and a first end of the upper rim and a second end of the upper rim define an opening from 30 degrees to 180 degrees. In one aspect, the first diameter is the same as the second diameter. In another aspect, the first diameter is greater than the diameter of the corneal limbus of a procedure eye. In still another aspect, the first diameter is less than the second diameter. In another alternative, the lower rim also has a lower surface having a curvature, a contour or a durometer adapted and configured to be secured to the sclera and/or conjunctiva of a procedure eye.

Additionally or optionally, the lower surface of the upper rim is adapted and configured for releasable engagement with an upper surface of the lower rim. There are also variations where an opening between the central portion and the first end of the upper rim or between the central portion and the second end of the upper rim. Still further, there may also be provided a breakaway coupling allowing the suction surface to remain coupled to the procedure eye while other portions of the docking device and system may be moved away.

In one embodiment, there is a breakaway coupling positioned between a position arm and an upper rim of the docking device, between an upper rim of the docking device and an upper portion of a sidewall of the docking device, or between a lower rim of the docking device and a lower portion of a sidewall of the docking device.

The various embodiments of the docking device and system may also be used to provide an advantageous set of methods for stabilizing and hydrating a procedure eye. In one aspect, there is a method of stabilizing a procedure eye during an ophthalmic surgical procedure by positioning a docking device having a lower surface with a suction ring on the procedure eye such that a central portion of the suction ring is at, on, or adjacent to a plica semilunaris and a lacrimal caruncle of the procedure eye and a portion of an opening in the suction ring is opposite to the central portion, the opening ranging from 30 degrees to 180 degrees. Next, applying vacuum to the suction ring to secure the suction ring lower surface to the surface of the sclera and/or conjunctiva, the suction ring at least partially encircling the cornea of the eye. Thereafter, performing the ophthalmic surgical procedure on the procedure eye using a surgical instrument passed through the opening in the suction ring. In one variation, there is also a step of hydrating the eye while the docking device is attached to the procedure eye. In other variations, there may also be a step of operating a positioning arm of an ophthalmic imaging and illumination system prior to the positioning step and the applying the vacuum step so that the suction ring is in alignment for use with the ophthalmic imaging and illumination system and for providing access to the procedure eye using the opening in the suction ring.

Additionally or optionally, prior to the step of operating a positioning arm step, there is a step of releasably coupling the docking device to the positioning arm. In another variation, the breakaway coupling and other features of the docking device may be implemented such that if the patient head moves during the ophthalmic surgical procedure that movement may be translated into an uncoupling action to disengage the suction ring from the positioning arm. In various embodiments, this disengagement is accomplished using a breakaway coupling that is integrated into the docking device or system. In some embodiments, the suction ring remains coupled to the procedure eye after operation of the breakaway coupling. Still further variations are contemplated such as the breakaway coupling being placed between the position arm and an upper rim of the docking device, between an upper rim of the docking device and an upper portion of a sidewall of the docking device, or between a lower rim of the docking device and a lower portion of a sidewall of the docking device. Optionally, there may also be provided steps for hydrating the procedure eye using an irrigation system coupled to the positioning arm.

### BRIEF DESCRIPTION OF THE DRAWINGS

Various characteristics of the non-limiting embodiments disclosed and described herein may be better understood by reference to the accompanying figures, in which:

FIG. 1 is a perspective view of a docking device having a suction ring, and a support ring. There is also a hydration nozzle adjacent to the support ring. A suction port is also shown that is in communication with the suction ring.

FIG. 2 is a perspective view of a docking device having a suction ring and a support ring similar to that in FIG. 1. In contrast to FIG. 1, FIG. 2 illustrates a hydration nozzle outlet that is integral to the support arm and support arm assembly. The suction port is attached to the suction tube and is not visible in this view.

FIG. 3A is an upper top perspective view of a prototype docking device in position on a cadaver eye. The lower rim adjacent to the high rim has a smaller diameter than the upper rim. Additionally, two openings in the rim sidewall are visible in this view adjacent to the central portion. The angle of the opening between the first end and the second end of the rim is visible in this view.

FIG. 3B is a right front view of the prototype docking device in position on a cadaver eye of FIG. 3A. This view provides an additional perspective on the relative positions of the central portion to the edges of the rim to delineate an opening for surgical access to the eye stabilized by the suction ring.

FIG. 3C is a top view over the opening between the first end and the second end of the rim of the prototype docking device in position on a cadaver eye of FIG. 3A. The angle of the opening provided for surgical access is clearly shown in this view. Additionally, the possible additional access points via the first and the second rim sidewall openings are also shown in this view.

FIG. 4 is a top-down illustration of the prototype in position on a cadaver eye as in FIGS. 3A-3C shown in phantom with the remaining surrounded eye anatomy added around the cadaver eye. In this view, the relationship between the central portion to the corner of the eye as well as the first and second ends to the angle of the opening are also shown.

FIG. 5A is a side view of a variation of the docking device of FIG. 3A-3C.

FIG. 5B is a perspective view of the support ring above the top surface of the upper rim of the docking device prior to engagement. The position and alignment of the detent features in the upper surface of the upper rim are visible in this view.

FIG. 5C is a bottom-up view of the view of FIG. 5B showing the detail of the mating features on the bottom surface of the support ring.

FIG. 6 is a perspective view of an embodiment of a docking device in position on the eye and coupled to a positioning arm below the position of representative imaging and illumination components. The areas of surgical access provided by the opening of the docking device are indicated and also visible in this view.

FIG. 7A is a view from the top of a patient's head who is prepared for a surgical procedure using the docking device as shown in FIG. 6. This view shows the relationship of a forehead-worn push stick that is in contact with some part of the positioning arm.

FIG. 7B is the top of the patient's head view of FIG. 7A showing the result of decoupling of the docking device when movement of the patient's head urges the positioning arm out of the docked position thereby disengaging the docking device.

FIG. 8A is a side view of a suction ring attached to a procedure eye with a rim and support ring adjacent and approaching the upper surface of the support ring.

FIG. 8B is a side view of a suction ring attached to a procedure eye as in FIG. 8A with a rim and support ring in contact with but misaligned with the upper surface of the support ring.

FIG. 8C is a side view of a suction ring attached to a procedure eye as in FIG. 8A with a rim and support ring in contact with and engaged with and aligned to the upper surface of the support ring.

FIG. 9 is a flow diagram illustrating a method of stabilizing an eye during an ophthalmic procedure.

FIG. 10 is a flow diagram illustrating a method of an ophthalmic docking system response to disruptive events.

### DETAILED DESCRIPTION

The various alternative embodiments described herein provide examples of intraocular docking devices that simultaneously provide: (1) visualization (2) eye stabilization and (3) surgical tool access. Additionally, each embodiment provides a clear line of sight to imaging and illumination systems. Stabilization systems are also described that provide adequate structural engagement to stabilize the eye yet efficiently position the docking structure with consideration for surgical tool workflow, operating envelopes including tool positioning trajectory and movement for each tool used during the surgical procedure. Additionally, the various docking device embodiments enable eye hydration to maintain moisture of the eye surfaces as well as a fluid barrier to assist in maintaining the visual field. Hydration may be provided manually or as part of an onboard hydration device as further detailed below.

For example, there is a docking device for intraocular surgery, including: an upper rim having a second diameter; a lower rim having a first diameter and a bottom surface with at least one aperture; an arm having a first portion coupled to a central portion of the upper rim and the lower rim and a second portion configured for connection to a positioning arm; and a lumen within or along the arm in communication with the at least one aperture; in which a first end of the upper rim and a second end of the upper rim define an opening from 30 degrees to 180 degrees.

According to one example of the docking device, the first diameter is the same as the second diameter.

According to one example of the docking device, the first diameter is greater than the diameter of the corneal limbus of a procedure eye.

According to one example of the docking device, the first diameter is less than the second diameter.

According to one example of the docking device, the lower rim further includes a lower surface having a curvature, a contour or a durometer adapted and configured to be secured to the sclera and/or conjunctiva of a procedure eye.

According to one example of the docking device, a lower surface of the upper rim is adapted and configured for releasable engagement with an upper surface of the lower rim.

According to one example of the docking device, there is an opening between the central portion and the first end of the upper rim or between the central portion and the second end of the upper rim.

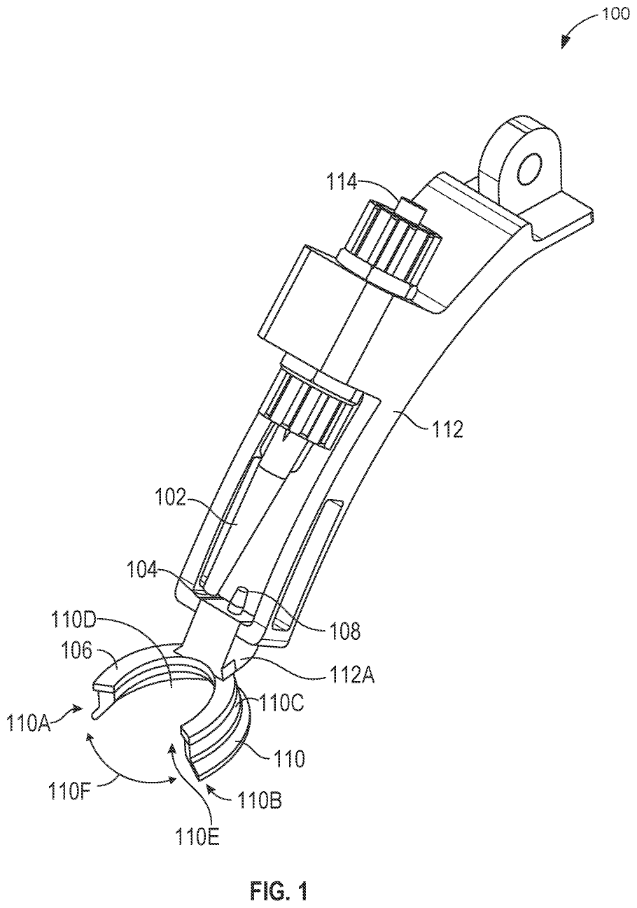

FIG. 1 is a perspective view 100 of a docking device having a suction ring, and a support ring. There is also a hydration nozzle 102 with nozzle outlet 104 adjacent to the support ring 106. A suction port 108 is also shown that is in communication with the suction ring 110. FIG. 1 shows the main features of the docking device 100. The suction ring 110 connects with the eyeball and, through suction force, secures the eyeball to the suction ring 100. This, in turn, secures the eyeball to the docking device 100 itself, fixing the eye in place. For support of the suction ring 110, the support ring 106 adds structural stability to the suction ring and connects with the support arm 112. The support arm 112 in turn interfaces with an external positioning device for assisting in the positioning of the overall docking device to a patient's eyeball. The suction ring 110 receives its suction through the suction port 108. Also shown is connection 112A between support arm 112 and support ring 106.

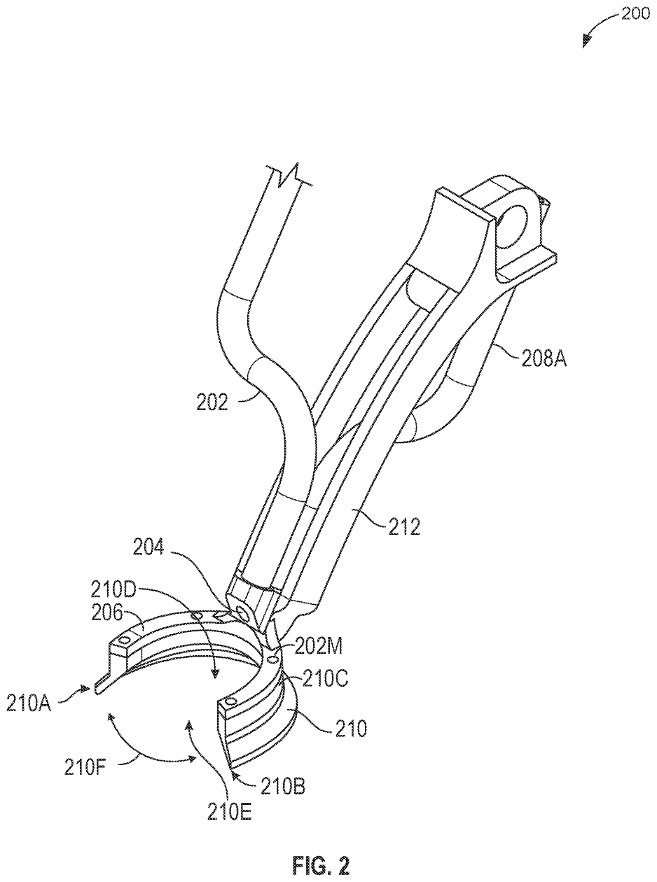

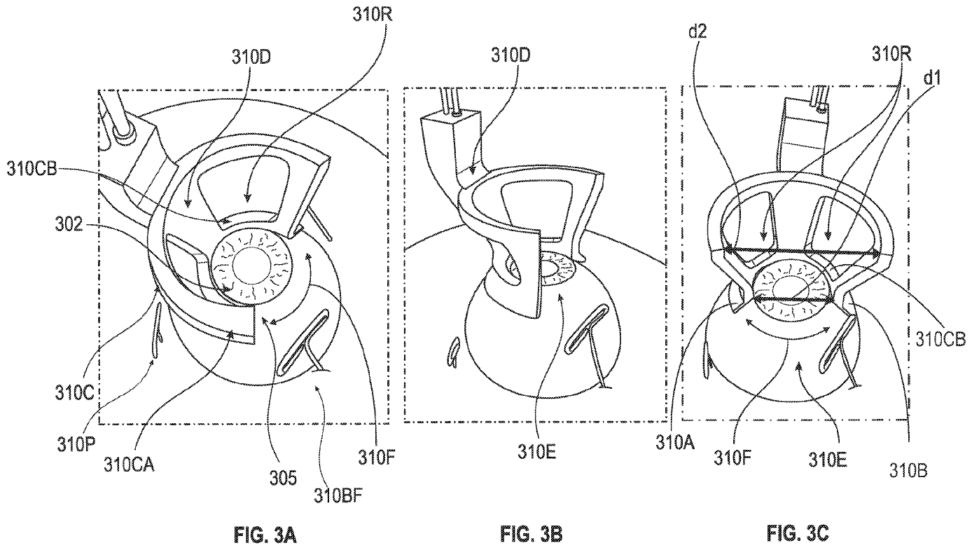

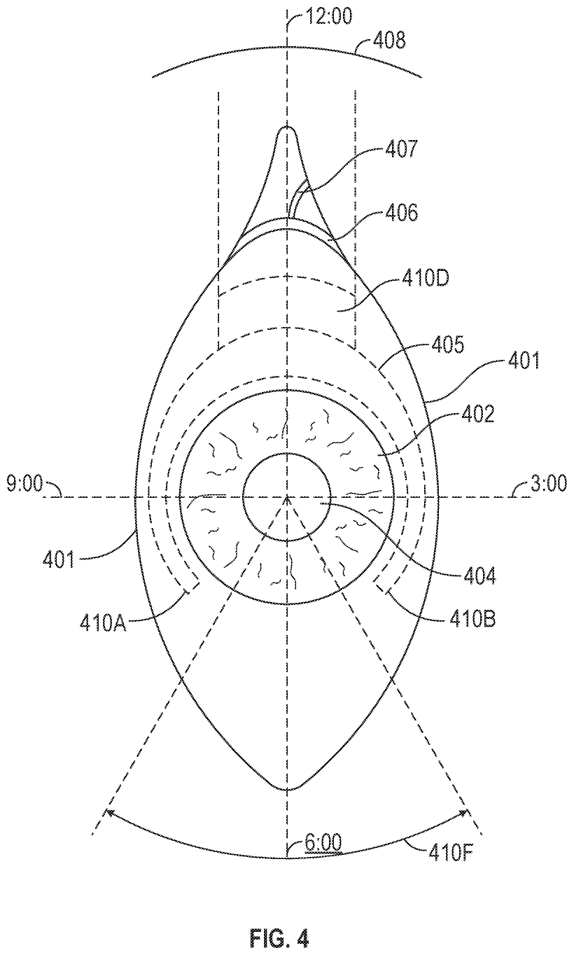

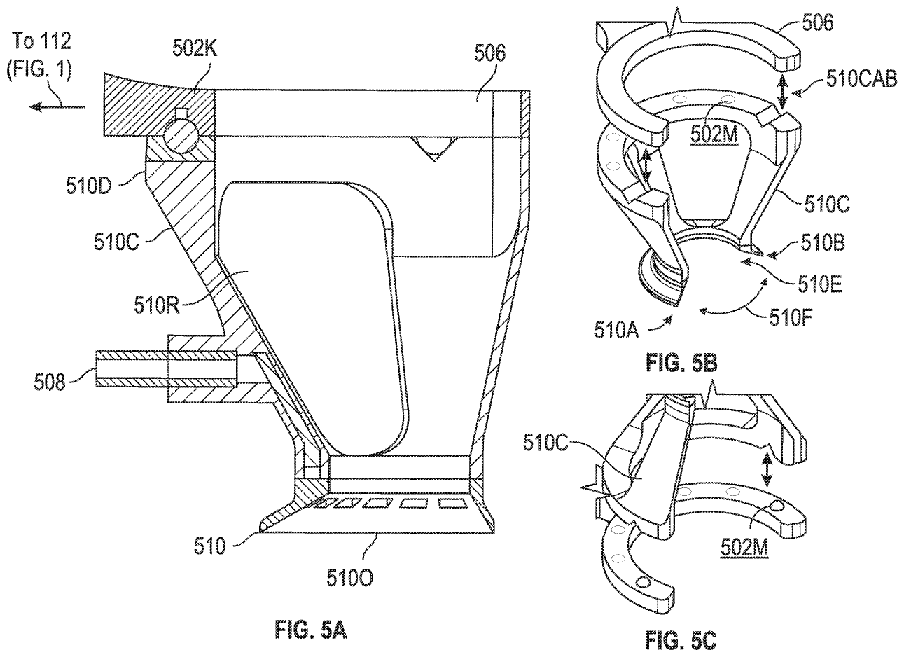

In one aspect, there is provided an embodiment of the present invention that enables the simultaneous use of the docking device 100 alongside surgical instruments operating inside the eye. Many conventional designs thwart this combination use because of either or both of solid rim and fully encircling docking design aspects. In contrast, embodiments of the inventive docking device 100 may have partial encirclement designs, selective encirclement designs, as well as open rim sidewall designs that allow for additional surgical tool access or for use of accessories as described herein. Still further, embodiments of the docking device advantageously configure aspects of a partial “ring” design that secures the eyeball on the nasal side while leaving the temporal side of the eye open and accessible to tools. (See FIG. 4). The docking device includes, in a variety of aspects, an upper rim for coupling to a support arm and various accessories (see FIGS. 1, 2, 3A-3C, 6, 7A and 7B) and a lower rim adapted and configured for coupling to a procedure eye in a range of different configurations (see FIGS. 1, 2, 3A-3C, and 5A-5C). A wide range of different shapes and orientations of the lower rim and upper rim are also appreciated with regard to the various configurations described with regard to FIG. 4. Additionally or optionally, the sidewall that extends between and joins the upper rim to the lower rim may be a continuous sidewall as seen best in FIGS. 1 and 2 or with a discontinuous or open sidewall construction such as shown in FIGS. 3A-3C and FIGS. 5A-5C.

FIG. 1 illustrates an exemplary partial suction ring 110/support ring 106 having a first end 110A, second end 110B, rim wall 110C, central portion 110D, opening 110E, and an angle of opening 110F.

In another embodiment, the docking device may incorporate an active hydration system to maintain a fluid layer and ensure adequate hydration during surgical procedures.

There may be a hydration port 114 coupled to an external nozzle (not shown) as in FIG. 1.

Optionally, the hydration outlet may be integral to support ring or other structure and coupled to appropriate tubing as in FIG. 2. FIG. 2 is a perspective view 200 of a docking device having a suction ring and a support ring similar to that in FIG. 1. In contrast to FIG. 1, FIG. 2 illustrates a hydration nozzle outlet 204 that is integral to the support arm 212 and support arm assembly. The suction port is attached to the suction tube 208A and is not visible in this view.

The docking device hydration function is similar to that of an assistant surgeon or nurse during traditional surgical procedures, who maintain corneal hydration through use of a hydrating syringe and cannula. The fluid can be balanced salt solution (BSS), medication, or other hydrating fluids.

One variation is the integrated hydration nozzle/hydration tube 202 as shown in FIG. 2. Another variation is to secure commercially available cannulas and/or nozzles to the docking system. Another variation is to integrate dedicated hydration ports into the structural docking ring and/or suction ring; either from one side, all sides, or some combination of the two. Also shown are coupling devices on support ring 206 such as magnets 202M. Also shown are first end 210A, second end 210B, rim wall 210C, central portion 210D, opening 210E, and an angle of opening 210F.

FIG. 3A is a nearly top perspective view of a prototype docking device in position on a cadaver eye. The lower rim 310CB adjacent to the eye has a smaller diameter than the upper rim 310CA. Additionally, two openings in the rim sidewall 310R are visible in this view adjacent to the central portion 310D. The angle of the opening 310F between the first end 310A and the second end 310B of the rim 310C is visible in this view. Pins or other securing devices 310P may assist in securing or stabilizing the cadaver eye to a base floor 310BF or other surface, or to affix a covering upon which the cadaver eye rests.

FIG. 3B is a right front view of the prototype docking device in position on a cadaver eye of FIG. 3A. This view provides an additional perspective on the relative positions of the central portion 310D to the edges of the rim 310C to delineate an opening 310E for surgical access to the eye stabilized by the suction ring 110 (from FIG. 1).

FIG. 3C is a top view over the opening between the first end 310A and the second end 310B of the rim 310C of the prototype docking device in position on a cadaver eye of FIG. 3A. The angle of the opening 310F provided for surgical access is clearly shown in this view. Additionally, the possible additional access points via the first and the second rim sidewall openings 319R are also shown in this view.

According to certain examples, the diameter of lower portion of rim 310CB is adapted and sized to be wider than the cornea 302 when in use. In this view on a cadaver eye, full cornea 302 and an amount of a portion of the sclera 305 around cornea 302 is seen. Angle of the opening 310F between the first end 310A and the second end 310B of the rim 310C is also indicated. In an additional aspect, the opening 310E is selected to provide a range of different approach angles to the corneal limbus 302.

FIG. 4 is a top-down illustration 400 of the prototype in position on a cadaver eye as in FIGS. 3A-3C shown in phantom (dotted lines) with the remaining surrounded eye anatomy added around the cadaver eye. In this view, the relationship between the central portion 410D to the corner of the eye as well as the first 410A and second 410B ends to the angle of the opening 410F are also shown. Also shown is the relationship of the lower rim diameter d1 (from FIG. 3C) which spans along a straight line, for example a straight line between 3:00 and 9:00, to the overall visibility of at least the entire iris and/or cornea 402 when the suction ring 110 (from FIG. 1) is coupled to the surface of the sclera 405. As such, it is to be appreciated that variations of the present invention may touch all, some, or none of the cornea; or all, some, or none of the sclera 405/conjunctiva. Also shown is the border between the sclera/conjunctiva and the eyelid 401, as well as pupil 404.

In use, the docking device central portion 410D at least partially covers the plica semilunaris 406 and lacrimal caruncle 407 (i.e., corner of the eye) or a portion of the sclera 405 or conjunctiva. When the suction ring 110 (from FIG. 1) bottom surface engages the sclera 405/conjunctiva, there is substantially all of the cornea 402 visible or all of the cornea 402 and a ring of sclera 405 between the cornea 402 and the inner edge of the suction ring 110 (from FIG. 1). The corneal limbus may be accessed via the opening.

The opening that allows access for the procedure is defined as that region between the first end 410A and the second end 410B of the suction ring 110 (from FIG. 1). Using a clock face with 12:00 (12 o'clock) at the central portion 410D near the corner of the procedure eye 407, then the 6:00 (6 o'clock) is on the opposite corner of the procedure eye. For an embodiment where the first end is at 9:00 (9 o'clock) and the second end is at 3:00 (3 o'clock), then the opening angle 410F is 180 degrees. If the ends are at 4 and 8 then the opening extends from 4-5-6-7-8. Similarly, if the ends are at 5 and 7 then the opening extends from 5-6-7. The ends need not be symmetrically spaced so that the opening is evenly spaced about the 6:00 (6 o'clock) position. Depending on the position and approach angle for an intraocular procedure, the opening and the engagement surface may be adjusted to provide the opening in an optimal approach angle.

Alternatively, in some embodiments, the central portion 410D remains positioned over the corner of the eye nearest the nose 408 but the opening defined by the positioning of the first end 410A and the second end 410B of the suction rim wall 110C (from FIG. 1) may be positioned in other locations not symmetrical to the 6:00 (6 o'clock) position as shown. Other docking devices may have the opening positioned in support of a different desired surgical approach vector to the eye stabilized by the suction ring 110 (from FIG. 1).

FIG. 5A is a side view of a variation of the docking device of FIGS. 3A-3C. As shown here, there is a kinematic ball coupling 502K which according to certain examples may be positioned to system arm 112 (from FIG. 1). There are also apertures 5100 in suction ring 510 for vacuum attachment to the surface of the eye. Also shown is suction 508, sidewall 510C, upper portion of sidewall 510D, and sidewall aperture 510R.

FIG. 5B is a perspective view of the support ring 506 above the top surface of the upper rim (310CA from FIG. 3A) of the docking device prior to engagement. The position and alignment of the detent features in the upper surface of the upper rim 510CAB are visible in this view. Also shown is first end 510A, second end 510B, sidewall 510C, opening 510E, and angle of opening 510F.

FIG. 5C is a bottom-up view of the view of FIG. 5B showing the detail of the mating features on the bottom surface of the support ring, such as for magnetic coupling 502M.

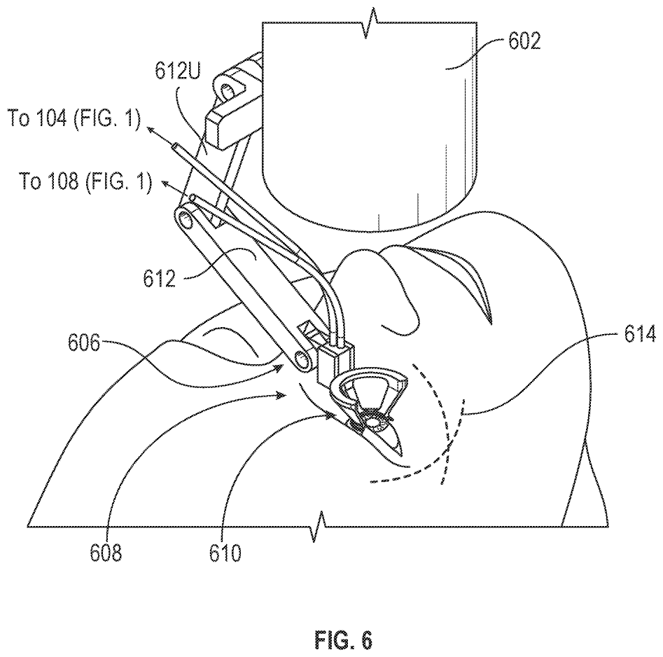

FIG. 6 is a perspective view of an embodiment of a docking device 610 in position on the eye and coupled to a positioning arm 612 below the position of representative imaging and illumination components 602. The areas of surgical access 614 provided by the opening of the docking device 610 are indicated and also visible in this view. Also shown is exemplary coupling 606 between docking device 610 and positioning arm 612, as well as rim opening access 608. Also shown is an upper arm or positioning arm 612U.

Advantageously, embodiments of the present invention are specifically designed to allow for increased visualization of the eye from a wide range of viewing angles while also facilitating illumination to the eyeball. In some embodiments, the docking device is adapted and configured to optimize visualization inside the eye while not blocking or obstructing the microscope or OCT view. (See the views of FIGS. 3A-3C and FIG. 6). Likewise, the docking device is specifically designed to optimize external illumination by virtue of its “open” design as is shown in FIG. 6 in relation to the exemplary illumination and imaging system. The openings in the sidewall between the upper and lower rim is useful in this regard. In additional alternative embodiments, any of a wide array of illumination sources may be integrated into or attached to portions of the docking device in any location suited to that purpose.

In some embodiments of the present invention there may be incorporated into the overall procedure in the use of the docking device a passive method to account for patient head and/or eye motion. In some aspects, the docking device is secured to the visualization system, but the design allows for and/or facilitates dislocation/motion of the docking itself in the event of patient motion. This could be done for, among other reasons, safety, or improving visualization of the intraocular workspace.

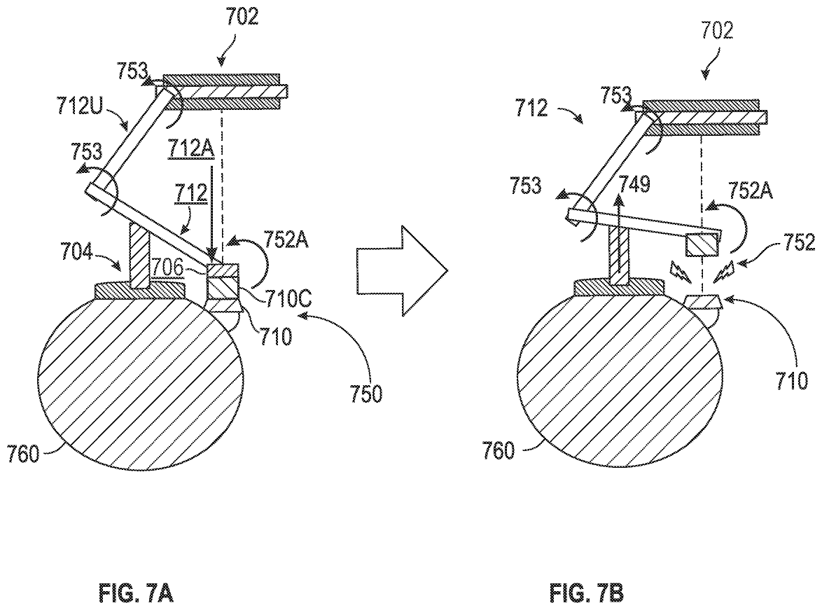

In one variation on passive actuation motion/response, which do not require active sensing or motor input to function, there is a push stick as shown in FIGS. 7A and 7B.

FIG. 7A is a view from the top of a patient's head who is prepared for a surgical procedure using the docking device as shown in FIG. 6. This view shows the relationship of a forehead worn push stick 702 that is in contact with some part of the positioning arm 712. As shown here, there is a procedure eye (of a patient's head 760) with docking device attached 750. In one example, the forehead pad with push stick 704 may not be coupled to system arm 712. In other examples, the forehead pad with push stick 704 is coupled to system arm 712, or support arm 112 (from FIG. 1), which may be in turn coupled to system arm 712. According to certain examples, the docking device is coupled to the exemplary positioning system arm 712 and moving 712A the docking device 752A attached to the procedure eye may move 753 the positioning system arm 712. Also shown is support ring 706, suction ring 710, and rim wall 710C. System arm 712 may be coupled to a positioning arm 712U which may be coupled to other components 702 which may be other connections, imaging systems, consoles, robotic systems, etc.

FIG. 7B is the top of the patient's head view of FIG. 7A showing the result of decoupling 752 of the docking device when movement of the patient's head urges the positioning arm out of the docked position 752 thereby disengaging 752 the docking device. According to certain examples, disengaging 752 or moving 752A the docking device may move the system arm 753.

In the embodiments of FIGS. 7A and 7B, the external “push stick” is mounted to the patient's head (for example via a forehead pad) 704 and is in contact with the docking arm 712. In the event the patient's head moves forward 749, the push stick 704 pushes the docking arm 712 out of the way and automatically decouples the docking device from the patient's eye (for example, decoupling from the suction ring 752).

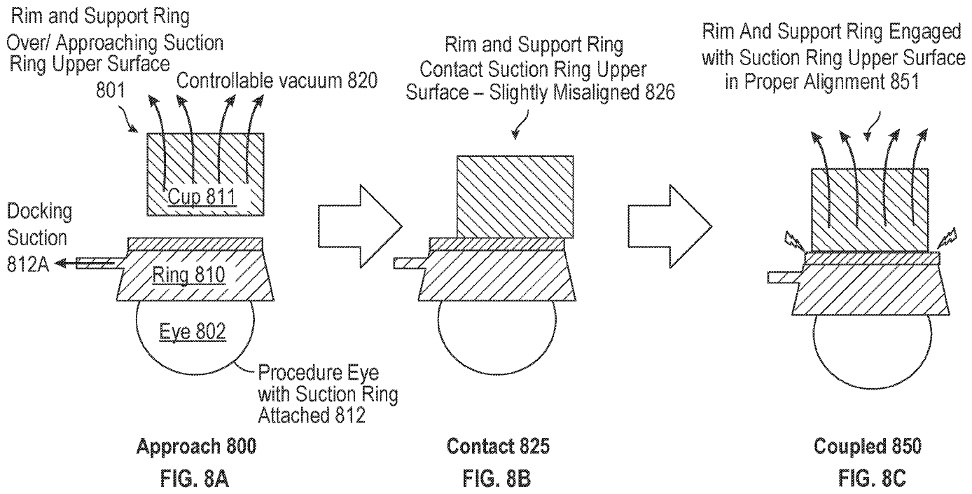

As an important component of the ability to quickly connect/disconnect the docking from its structural support elements, another embodiment incorporates a variety of means of accomplishing said requirement. This repeatable process and capability may be considered in a three step process of approach, contact and coupling as detailed below. Once coupled for use in the surgical procedure, uncoupling may take place by an automatic process, a manual process or a de-coupling process as shown in FIGS. 7A and 7B. It is to be appreciated that coupling forces used between and among the different components of the docking device and associated portion of the positioning arm may be mechanical, magnetic, pneumatic, vacuum-based, or the like. For illustrative purposes, FIGS. 8A, 8B and 8C represent a schematic version provided to explain a vacuum-based design variation.

FIG. 8A is a side view of a suction ring attached to a procedure eye with a rim and support ring adjacent and approaching the upper surface of the support ring 800. As shown here, there is an approach phase 800 in which rim and support ring go over/approach 801 the upper surface of the suction ring 810. Here, the procedure eye 802 with suction ring 810 may be secured together 812 via docking suction 812A. Also shown is cup 811 with a controllable vacuum 820 applied and the rim wall 110C (from FIG. 1) and support ring 106 (from FIG. 1) move over or approach the suction ring 810 upper surface.

FIG. 8B is a side view of a suction ring attached to a procedure eye as in FIG. 8A with a rim and support ring in contact with but misaligned with the upper surface of the support ring 825. As shown here, there is a contact phase 825 in which the rim wall 110C (from FIG. 1) and support ring 106 (from FIG. 1) are in contact with the upper surface of the support ring but are slightly misaligned 826. According to certain examples, such misalignment is acceptable.

FIG. 8C is a side view of a suction ring attached to a procedure eye as in FIG. 8A with a rim wall 110C (from FIG. 1) and support ring 106 (from FIG. 1) in contact with and engaged with and aligned to the upper surface of the support ring 850. As shown here, there is a coupling phase 850, in which the rim wall 110C (from FIG. 1) and support structure 106 (from FIG. 1) are engaged with the suction ring 810 upper surface, in proper alignment 851 for surgery to proceed. Coupling process or engage coupling 850 ensures proper alignment 851 between rim wall 110C bottom surface and suction ring 810 upper surface. According to certain examples, different levels of coupling forces are possible.

In addition to the coupling force, the docking device may incorporate kinematic coupling elements which ensure the docking attaches to the same location during attachment. These couplers can either be precision “kinematic couplings” or some other physical, active, or passive means. Their incorporation into the docking device ensures that the coupling will be precise, i.e., coupling into a near-identical position every time. FIGS. 5A, 5B and 5C provide one type and arrangement of a coupling approach.

FIG. 5A is a side view of a variation of the docking device of FIG. 3A-3C. The mating of the kinematic ball coupling 502K between the bottom surface of the support ring 506 and the upper rim (310CA from FIG. 3A) is shown in this view. FIG. 5B is a perspective view of the support ring 506 above the top surface of the upper portion of rim 510C of the docking device prior to engagement. The position and alignment of the detent features in the upper surface of the upper rim 510CAB are visible in this view. FIG. 5C is a bottom-up view of the view of FIG. 5B showing the detail of the mating features on the bottom surface of the support ring, such as for magnetic coupling 502M. Considered together, it is to be appreciated that there may be a range of different breakaway coupling configurations in various alternative docking device embodiments. In one embodiment, the breakaway coupling is between the upper rim and an upper portion of the sidewall as shown in FIGS. 5A and 5B. In yet another configuration, the breakaway coupling may be between a lower portion of the side wall and the lower rim as shown in FIG. 5C. In one aspect, the breakaway coupling in positioned so that upon actuation the portion of the docking device coupled to the eye remains coupled to the eye and the docking device separates by operation of a breakaway coupling in another location. As such, there is still another breakaway coupling variation that may be located at the connection point between the support arm and the upper rim, see for example where such a breakaway coupling may be located at the junction adjacent to the upper rim as indicated by 310D in FIG. 3B.

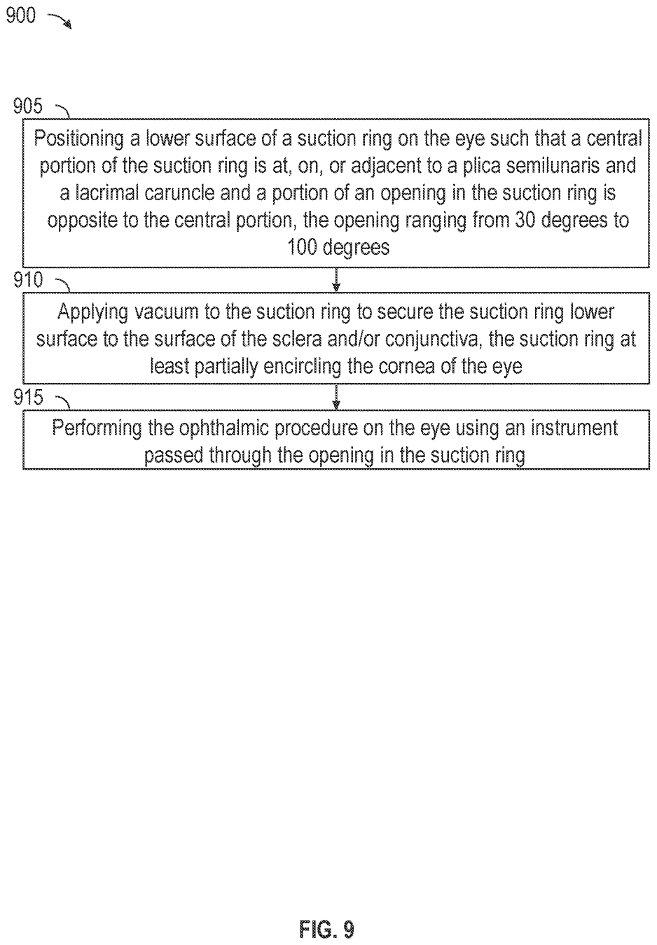

FIG. 9 is a flow diagram illustrating a method 900 of stabilizing an eye during an ophthalmic procedure.

Method 900 begins at block 905 with positioning a lower surface of a suction ring on the eye such that a central portion of the suction ring is at, on, or adjacent to a plica semilunaris and a lacrimal caruncle and a portion of an opening in the suction ring is opposite to the central portion, the opening ranging from 30 degrees to 180 degrees.

Method 900 continues at block 910 with applying vacuum to the suction ring to secure the suction ring lower surface to the surface of the sclera and/or conjunctiva, the suction ring at least partially encircling the cornea of the eye.

Next, at block 915, method 900 continues with performing the ophthalmic procedure on the eye using an instrument passed through the opening in the suction ring.

According to an embodiment of method 900, method 900 further includes operating a positioning arm of an ophthalmic imaging and illumination system prior to the positioning step and the applying the vacuum step so that the suction ring is in alignment for use with the ophthalmic imaging and illumination system and for providing access to the eye using the opening in the suction ring.

According to an embodiment of method 900, method 900 further includes: prior to the operating a positioning arm step, releasably coupling the suction ring to the positioning arm.

According to an embodiment of method 900, method 900 further includes moving a patient head during the ophthalmic procedure to uncouple the suction ring from the positioning arm.

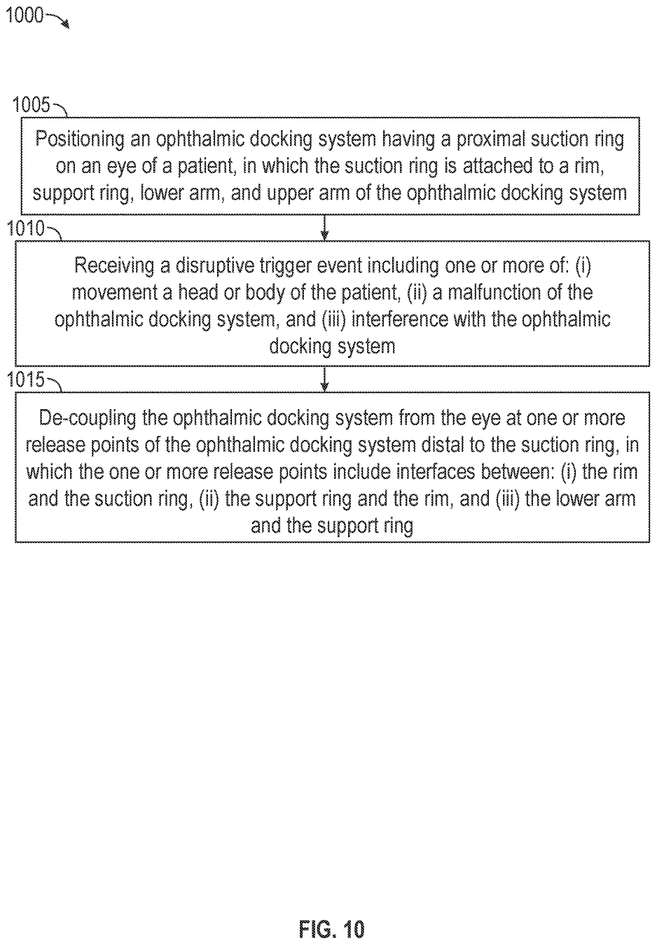

FIG. 10 is a flow diagram illustrating a method of a docking system response to disruptive events during ophthalmic procedures 1000, for example during eye surgery.

At block 1005, the method begins with positioning an ophthalmic docking system having a proximal suction ring on an eye of a patient, in which the suction ring is attached to a rim, support ring, lower arm, and an upper arm of the ophthalmic docking system.

Method 1000 continues at block 1010, with receiving a disruptive trigger event including one or more of: (i) movement of a head or body of the patient, (ii) a malfunction of the ophthalmic docking system, and (iii) interference with the ophthalmic docking system.

Method 1000 concludes at block 1015 with de-coupling the ophthalmic docking system from the eye at one or more release points of the ophthalmic docking system distal to the suction ring, in which the one or more release points include interfaces between: (i) the rim and the suction ring, (ii) the support ring and the rim, and (iii) the lower arm and the support ring.

In still other alternative embodiments, the docking device described herein may be adapted and configured for integration or exchangeable coupling with a variety of sensors to detect, measure or sense a variety of measurable parameters related to the function or performance of a component or assembly of a surgical tool or, additionally or optionally, to forces, pressures, torques, humidity, stress, temperature, and the like within the surgical field. In various alternative configurations, the sensors can either be embedded in the docking system/structure itself, or incorporated on the external faces to facilitate easy access or sensing ability.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A docking device for intraocular surgery, comprising:
an upper rim having a second diameter;
a lower rim having a first diameter and a bottom surface with at least one aperture;
a sidewall extending between the upper rim and the lower rim;
an arm having a first portion coupled to a central portion of the upper rim and the lower rim and a second portion configured for connection to a positioning arm; and
a lumen within or along the arm in communication with the at least one aperture;
wherein a first end of the upper rim and a second end of the upper rim define an opening from 30 degrees to 180 degrees.

2. The device of claim 1, wherein the first diameter is the same as the second diameter.

3. The device of claim 1, wherein the first diameter is greater than the diameter of the corneal limbus of a procedure eye.

4. The device of claim 1, wherein the first diameter is less than the second diameter.

5. The device of claim 1, the lower rim further comprising a lower surface having a curvature, a contour or a durometer adapted and configured to be secured to the sclera and/or conjunctiva of a procedure eye.

6. The device of claim 1, wherein a lower surface of the upper rim is adapted and configured for releasable engagement with an upper surface of the lower rim.

7. The device of claim 1, further comprising an opening between the central portion and the first end of the upper rim or between the central portion and the second end of the upper rim.

8. The device of any of claims 1-7, further comprising a breakaway coupling positioned between a position arm and an upper rim of the docking device, between an upper rim of the docking device and an upper portion of a sidewall of the docking device, or between a lower rim of the docking device and a lower portion of a sidewall of the docking device.

9. A method of stabilizing a procedure eye during an ophthalmic surgical procedure, comprising:
positioning a docking device having a lower surface with a suction ring on the procedure eye such that a central portion of the suction ring is at, on, or adjacent to a plica semilunaris and a lacrimal caruncle of the procedure eye and a portion of an opening in the suction ring is opposite to the central portion, the opening ranging from 30 degrees to 180 degrees;
applying vacuum to the suction ring to secure the suction ring lower surface to the surface of the sclera and/or conjunctiva, the suction ring at least partially encircling the cornea of the eye; and
performing the ophthalmic surgical procedure on the procedure eye using a surgical instrument passed through the opening in the suction ring.

10. The method of claim 9, further comprising hydrating the eye while the docking device is attached to the procedure eye.

11. The method of claim 9, further comprising: operating a positioning arm of an ophthalmic imaging and illumination system prior to the positioning step and the applying the vacuum step so that the suction ring is in alignment for use with the ophthalmic imaging and illumination system and for providing access to the procedure eye using the opening in the suction ring.

12. The method of claim 11, further comprising: prior to the operating a positioning arm step releasably coupling the docking device to the positioning arm.

13. The method of any one of claims 9, 11 or 12, wherein moving a patient head during the ophthalmic surgical procedure will uncouple the suction ring from the positioning arm using a breakaway coupling.

14. The method of claim 12, wherein the suction ring remains coupled to the procedure eye after operation of the breakaway coupling.

15. The method of claim 13, wherein the breakaway coupling is between the position arm and an upper rim of the docking device, between an upper rim of the docking device and an upper portion of a sidewall of the docking device, or between a lower rim of the docking device and a lower portion of a sidewall of the docking device.

16. The method of claim 11, further comprising hydrating the procedure eye using an irrigation system coupled to the positioning arm.

17. A method of docking system response to disruptive events during ophthalmic procedures, comprising:
positioning an ophthalmic docking system having a proximal suction ring on the eye of a patient, wherein the suction ring is attached to a rim, support ring, lower arm, and an upper arm of the ophthalmic docking system;
receiving a disruptive trigger event including one or more of: (i) movement of a head or body of the patient, (ii) a malfunction of the ophthalmic docking system, and (iii) interference with the ophthalmic docking system; and
de-coupling the ophthalmic docking system from the eye at one or more release points of the ophthalmic docking system distal to the suction ring, wherein the one or more release points include interfaces between: (i) the rim and the suction ring, (ii) the support ring and the rim, and (iii) the lower arm and the support ring.
